(19) United States
(12) Patent Application Publication
(10) Pub. No.: US 2021/0378523 A1
(43) Pub. Date: Dec. 9, 2021

(54) SENSOR UNIT FOR A MEDICAL SUPPORT SYSTEM FOR IMPLANTATION IN A PATIENT AND METHOD FOR PRODUCING A SENSOR UNIT

(71) Applicant: KARDION GMBH, Stuttgart (DE)

(72) Inventors: Martina Budde, Karlsruhe (DE); Thomas Alexander Schlebusch, Renningen (DE); Julian Kassel, Böblingen (DE)

(21) Appl. No.: 15/734,831

(22) PCT Filed: Jun. 6, 2019
(86) PCT No.: PCT/EP2019/064774

§ 371 Date: Jun. 18, 2021

(30) Foreign Application Priority Data
Jun. 6, 2018 (DE) 10 2018 208 916.7

(51) Int. Cl. A61B 5/0215; G01L 19/14

(52) U.S. Cl. CPC A61B 5/0215; A61B 2562/12; A61B 2562/0247; G01L 19/149

(57) ABSTRACT

The invention relates to a sensor unit (100) for an implant system for medical support of a patient, wherein the sensor unit (100) comprises a carrier material (110) in which a recess (120) is formed and, furthermore, the sensor unit (100) comprises a semiconductor component (130) for forming a sensor, wherein the semiconductor component (130) is arranged in the recess (120) and, lastly, the sensor unit (100) comprises a substrate layer (140), which covers at least partially the recess (120) and/or comprises an opening (150) on at least one side of the sensor unit (110), as well as a diffusion barrier, by means of which at least the semiconductor component (130) is at least partially covered or coated in order to ensure a medium access (150) to the sensor.

The invention is based on a device or a method as defined in the preamble of the independent claims. The subject matter of the present invention is also a computer program.

Many different types of ventricular assist systems are used; a device is selected that is best suited for the heart disease of the individual patient. A left ventricular or a right ventricular assist system is implanted in the patient, depending on the heart chamber to be supported.

Ventricular assist systems with integrated sensor technology are known. For example, the patent document US 2016101230 A1 describes a ventricular assist system with pressure sensors in inlet cannulas.

Based on the aforesaid, the object of the present invention is to provide a sensor that is simplified and improved in terms of its construction for an implant system as well as a suitable method for the production of said sensor.

Against this background, the approach presented here introduces a sensor unit for an implant system for medical support of a patient, a method for producing a sensor unit, furthermore a device that uses said method, and lastly a corresponding computer program according to the main claims. Advantageous further developments and improvements of the device disclosed in the independent claim are made possible by means of the measures listed in the dependent claims.

The present sensor unit for an implant system, for example a ventricular assist system for medical support of a patient, uses a novel construction and connection technique for increasing the stability and for miniaturizing medical sensor systems that are in direct contact with a tissue and/or a fluid of a patient.

Presented is a sensor unit for an implant system for medical support of a patient, wherein the sensor unit has the following features:
a carrier material in which a recess is formed;
a semiconductor component for forming a sensor with the semiconductor component being arranged in the recess;
a substrate layer that covers at least partially the recess and/or has an opening on at least one side of the sensor unit in order to ensure a medium access to the sensor; and
a diffusion barrier, by means of which at least the semiconductor component is at least partially covered or coated.

A sensor unit may be, for example, a composite consisting of a plurality of technical components and/or functional units, with which a generally electrical signal, for example a heart pressure signal, can be generated as a function of a physical and/or geometrical variable. Therefore, the sensor unit may be, for example, a medical sensor system of a temporarily and/or long-term implanted system, for example a pressure sensor for measuring the arterial pressure or pressure pulsation of a patient suffering from a heart disease. As an alternative, however, the sensor unit may also be any medical sensor system that is implanted in the human body. An implant system may be, for example, an artificial material system that is implanted in the body and is intended to remain in the body of the patient permanently and/or at least for a longer period of time. Therefore, implant systems can be differentiated, for example, according to whether they are medical, plastic, and/or functional implant systems, wherein the medical implant system may be, for example, an artificial heart and/or a cardiac pacemaker. A carrier material may be, for example, a bearing and/or supporting material that can be used for mechanically anchoring an electronic component. A recess may be, for example, a cut and/or a hollow shape in a surface, for example a carrier material and/or support material, wherein the recess can be used, for example, for receiving an electronic component. A diffusion barrier may be, for example, a fluid tight seal and/or membrane that can protect, for example, electrical contacts of an electronic component from penetrating fluids (body fluids, such as blood) or can protect said electrical contacts from the infusion of said body fluids and, in so doing, can protect them from short circuits, which are associated with such an infusion of body fluids. At the same time, such a diffusion barrier can ensure a medically qualified surface. In this respect, the semiconductor component can be at least partially sealed in a fluid-tight manner on the surface of said semiconductor component. A semiconductor component may be, for example, an electronic component that is used, for example, to form a sensor, for example a pressure sensor. In this case, the semiconductor component can be characterized by a material that is conductive only under certain physical and electrical conditions. Therefore, the semiconductor component can be produced, for example, on a silicon basis. A substrate layer may be, for example, a medically qualified, flexible polyimide printed circuit board and/or a rigid material, such as glass and/or silicon, which can be used, for example, to close a recess and/or can partially cover at least the recess. In this case, the substrate layer can have, for example, an opening and/or a perforation, wherein said substrate layer does not cover the recess, as a result of which a medium access to a semiconductor component can be provided. Furthermore, the substrate layer can also be, for example, in direct contact with a (human or animal) tissue and/or a fluid of the patient without damaging the tissue or the fluid or without itself being damaged by the tissue or the fluid. At the same time, the substrate layer can also implement, for example, the electrical contacting of the sensors and/or microcontrollers. A medium access may be an entry region and/or exit region for liquid and/or gaseous substances and/or substances and/or matter. In this respect the medium access can be produced, for example, by forming an opening in a substrate layer in order to provide a medium access to a sensor. In this case, the sensor may be, for example, an implanted pressure sensor that measures the heart pressure of a patient directly in the pulmonary artery, for which purpose a medium access is generally required.

The advantages of the medical sensor unit presented here lie in particular in the construction and connection technique that requires a medium access, depending on the requirement of the implant system. In this context, the sensor unit describes a novel variant of the design that offers, in particular for long implantation times, advantages in the area of a possible deviation of a sensor signal, for example a heart pressure sensor signal, as well as aging stability. In this case, a sensor with a medium access, optionally also without a medium access, is integrated in the medical sensor unit without requiring a multilayer structure between the sensor and a tissue and/or a fluid of a patient. Furthermore, in the production process of the sensor unit, there is no need for bonding wires, an aspect that ensures greater mechanical stability and less risk of damage to the implant system, for example during production. Consequently, there is of course no need to ensure, as customary, the mechanical protection of the bonding wires. A series production of the sensor unit presented here can be simplified, because further processing steps can be carried out during the production of the sensor unit while the silicone gel and/or silicone oil, which is/are still required for anchoring the sensor, cures.

In accordance with one embodiment, the carrier material can comprise metallic material, thermoplastic, and/or ceramic and/or glass. The metallic material that can be used may be, for example, titanium, NiTiNol, or stainless steel, which may be medically relevant carrier materials, in particular. Such an embodiment of the approach presented here offers the advantage that materials made of thermoplastic and/or ceramic and/or glass have high barrier properties. Concepts based on such materials can be integrated, on the one hand, directly in the material system of the sensor unit and/or, on the other hand, can also be fluidically connected to the semiconductor component, arranged in the carrier material by a joining process such as adhesively bonding, welding, and/or clamping.

In accordance with one embodiment, the semiconductor component can be electrically and/or mechanically connected to the substrate layer by means of at least one contacting bump or contacting cusp, in particular, with the at least one contacting bump being formed from a gold material. Such an embodiment of the approach presented here offers the advantage that the contacting bump, for which wire made of pure gold and/or a gold alloy is used, can be contacted with the substrate layer in an adhesively bonded, clamped, or welded manner and/or by any other similar contacting methods, so that it is possible to dispense with and/or to simplify the processing steps in the production of the sensor unit. In this context, another advantage of such a contacting method is the low thermal stress.

In accordance with one embodiment, the substrate layer can be designed so as to be in direct contact with a tissue and/or a fluid of the patient, in particular with the substrate layer being formed as a polyimide element and/or from a ceramic material, glass, and/or silicon. In this case, the substrate layer itself and/or a coating of the substrate layer, for example a diffusion barrier, can form a medically qualified surface of the sensor unit. Furthermore, the substrate layer can also have, depending on the requirement of the implant system, an opening and/or a perforation that produces the medium access above the sensor system. Such an embodiment of the approach presented here offers the advantage that, for example, a better heart pressure signal can be produced for pressure sensors. As an alternative, however, it is also possible to dispense with the opening and/or perforation.

In accordance with one embodiment, the recess can be filled with a silicone gel and/or silicone oil; and/or the semiconductor component can be at least partially surrounded by the silicone gel and/or silicone oil. In this case, the filling of the recess and/or the surrounding area of the semiconductor component with the silicone gel and/or silicone oil and/or a similar material can be carried out before the sensor unit is assembled. Such an embodiment of the approach presented here offers the advantage that the semiconductor component is decoupled from any possible stress and thus secondly, is also anchored or fixed mechanically inside the recess. However, it is also possible, depending on the requirement of the implant system, to dispense with such a stress decoupling and/or fixing.

In accordance with one embodiment, the semiconductor component that is inserted into the recess can correspond essentially to the size of the recess. In this case, an edge corresponding to no more than 10% of the extent of the semiconductor component can remain essentially open, in particular inside the recess. Such an embodiment of the approach presented here offers the advantage that, given a perfect match between the size of the recess and the size of the semiconductor component, the semiconductor component does not need any mechanical anchoring.

In accordance with one embodiment, the substrate layer may have a thickness in the range between 40 μm and 60 μm; and/or the at least one contacting bump may have a thickness in the range between 90 μm and 110 μm. During the production of a sensor unit, it is absolutely necessary to observe the structural height. In this case, bonding wires usually contribute to the height of the sensor unit between 150 μm and 250 μm. In addition, bonding wires should still be mechanically protected. However, each variant of the protection further increases the structural height of the sensor unit. Such an embodiment of the approach presented here offers the advantage that in the case of the flip chip structure of the sensor unit used here, the thickness of the substrate layer is, for example, 50 μm and the thickness of the contacting bumps is, for example, 100 μm, with the result, however, that the mechanical protection is already ensured. Furthermore, uniform layer thicknesses in this layer thickness do not and/or only very slightly change and/or influence a sensor signal, for example a heart pressure sensor signal, so that there is no need for a time-consuming calibration of the implant system.

Presented is a method for producing a sensor unit according to any one of the preceding claims, wherein the method comprises the following steps of:

providing a carrier material, a semiconductor component, a substrate layer, and a diffusion barrier; and
arranging the carrier material, the semiconductor component, the substrate layer, and the diffusion barrier in such a way that the sensor unit is produced.

In accordance with one embodiment, it is possible to fill the recess with a silicone gel and/or a silicone oil for mechanically anchoring the semiconductor component inside the recess in the step of arranging. In this case, it is possible to carry out the filling of the recess and/or the surrounding area of the semiconductor component with the silicone gel and/or silicone oil and/or a similar material before the assembly of the sensor unit. Such an embodiment of the approach presented here offers the advantage that the semiconductor component is decoupled from any possible stress and thus, secondly, is also anchored or fixed mechanically inside the recess. It is also possible to omit, depending on the requirement of the implant system, such a stress decoupling and/or fixing.

In accordance with one embodiment, it is possible to coat the semiconductor component and/or the substrate layer with a diffusion barrier in the step of providing. Such an embodiment of the approach presented here offers the advantage that such a coating provides a qualified medical surface, offers protection of the electrical contacts from penetrating fluid, and, as a result, from short circuits, and furthermore prevents fluid from entering the supporting material and/or carrier material of the sensor unit, whereas, if this were not the case, it could result in swelling of the material. The resulting mechanical stress could lead to a change in the sensor signal, for example a heart pressure sensor signal, and, in so doing, considerably restrict the way in which the implant system works. As a diffusion barrier, the semiconductor component of the sensor and/or the substrate layer can also be provided with a layer of parylene that is a few micrometers thick. Such an embodiment of the approach presented here offers the advantage that parylene coatings are, for example, very light, form a homogeneous layer thickness and are nevertheless robust against environmental influences. In addition, the material is biocompatible, as it contains neither solvents nor plasticizers. At the same time, both coating techniques can be reproduced as a complete encapsulation of the sensor unit in, for example, a silicone material.

The method presented here for producing a sensor unit can be implemented, for example, in software or hardware or in a mixed form of software and hardware, for example in a control device.

Furthermore, the approach presented here also provides a device that is designed to execute, trigger, and/or implement the steps of a variant of a method presented here for forming a sensor unit in corresponding apparatuses. The problem on which the invention is based can also be solved quickly and efficiently by means of this design variant of the invention in the form of a device.

For this purpose, the device may comprise at least one computing unit for processing signals or data, at least one memory unit for storing signals or data, at least one interface to a sensor or an actuator for inputting sensor signals from the sensor or for outputting data signals or control signals to the actuator, and/or at least one communication interface for inputting or outputting data that is embedded in a communication protocol. The computing unit may be, for example, a signal processor, a microcontroller, or the like, and the memory unit may be a flash memory, an EEPROM, or a magnetic memory unit. The communication interface can be designed to input or output data wirelessly and/or in a wired manner, wherein a communication interface that can input or output data in a wired manner can input said data from a corresponding data transmission line or can output said data into a corresponding data transmission line in, for example, an electrical or optical manner.

A device in the present case may be understood to mean an electrical device that processes sensor signals and, as a function thereof, outputs control signals and/or data signals. The device can have an interface that is configured in hardware and/or in software. In the case of a design in hardware, the interfaces can be, for example, part of a so-called ASIC system, which includes a wide range of functions of the device. However, it is also possible for the interfaces to be separate, integrated circuits or to consist at least partially of discrete components. In the case of a design in software, the interfaces can be software modules that are present, for example, on a microcontroller, in addition to other software modules.

Advantageous is also a computer program product or a computer program having program code that can be stored on a machine-readable carrier or storage medium, such as a semiconductor memory, a hard disk memory, or an optical memory and is used to execute, implement, and/or trigger the steps of the method in accordance with any one of the embodiments described above, in particular if the program product or program is executed on a computer or a device.

Exemplary embodiments of the approach presented here are shown in the drawings and explained in more detail in the following description. The drawings show:

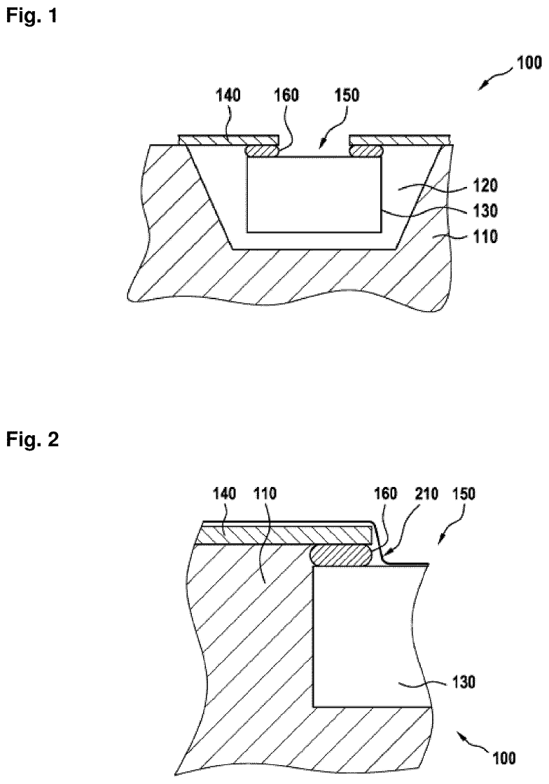

FIG. 1 in schematic form, a cross-sectional view of a sensor unit in accordance with one exemplary embodiment;

FIG. 2 section of a cross-section of a sensor unit in accordance with one exemplary embodiment in a schematic detail view; and

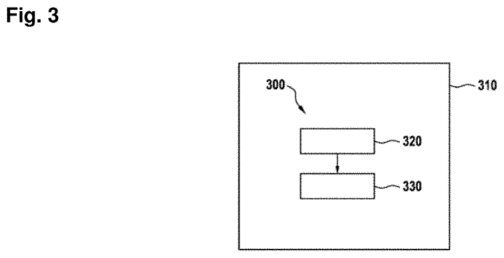

FIG. 3 flowchart of an exemplary embodiment of a method for producing a sensor unit in accordance with one exemplary embodiment.

In the following description of advantageous exemplary embodiments of the present invention, identical or similar reference numerals are used for the elements that are shown in the various figures and that act in a similar manner, and there is no need to repeat the description of these elements.

FIG. 1 shows in schematic form a cross-sectional view of a sensor unit 100 in accordance with one exemplary embodiment.

The sensor unit 100 has a carrier material 110, wherein a recess 120 is formed in the carrier material 110. In this respect, the recess 120 is formed in a parallel trapezoidal shape in accordance with one exemplary embodiment. A semiconductor component 130 for forming a sensor is arranged in the recess 120, wherein the semiconductor component 130 is shaped in a rectangular manner in accordance with one exemplary embodiment. In this context, a rectangular shape of the recess 120 and/or the semiconductor component 130 offers the advantage of a space-saving and cost-effective production of these components. In accordance with one exemplary embodiment, the recess 120 is made larger than the inserted semiconductor component 130, wherein an edge, which in accordance with one exemplary embodiment corresponds to no more than 10% of the extent of the semiconductor component 130, remains open inside the recess 120. Before the sensor unit 100 is assembled, the recess 120 is filled with a silicone gel and/or a silicone oil and/or a similar support material in order to decouple the semiconductor component 130 from mechanical stress and/or to mechanically anchor the same. However, such a stress decoupling or mechanical anchoring can also be omitted, depending on the requirement of the implant system. In so doing, the semiconductor component 130 is at least partially or completely covered or coated in an advantageous way with a diffusion barrier that will be described in greater detail below. The diffusion barrier is applied, for example, as a coat or layer that prevents a diffusion of body fluids into the sensor unit 100 or that prevents damage to parts of the sensor unit 100. For example, the diffusion barrier can also be deposited or formed by means of a final process step in a production process.

Furthermore, the sensor unit 100 comprises a substrate layer 140 that at least partially covers the recess 120, wherein the substrate layer 140 in one exemplary embodiment has an opening 150 and/or a perforation above the semiconductor component 130 in order to ensure a medium access to the sensor. The same reference numeral is used below for the opening 150 and the medium access 150. The substrate layer 140 is designed to be in direct contact with a tissue and/or a fluid of the patient. At the same time, the substrate layer 140 is used to make electrical contact with the sensors and microcontrollers. In accordance with one exemplary embodiment, the semiconductor component 130 is electrically and/or mechanically connected to the substrate layer 140 by means of two contacting bumps 160. In accordance with one embodiment, the two contacting bumps 160 have an elliptical shape. The diffusion barrier 210 can also cover or coat not only a surface or a portion of the surface of the semiconductor component 130 but can also completely coat or cover a portion of the substrate layer 140 or the substrate layer 140 itself.

As an alternative to the exemplary embodiment of a sensor unit 100 presented here, a sensor unit 100 can also be produced without a medium access 150, depending on the requirement of the implant system, wherein the structure of a sensor unit 100 without a medium access 150 is, in principle, identical to the structure of the sensor unit 100 shown here with a medium access 150.

In accordance with one exemplary embodiment, the sensor unit 100 shown here is produced by means of a so-called flip chip assembly. The flip chip assembly is a method of the construction and connection technology for contacting an unhoused semiconductor component 130 by means of at least one contacting bump 160. In the case of the flip chip assembly, a microchip (not shown) is mounted directly and without further connecting wires with the active contacting side facing the substrate layer 140. This arrangement leads to extremely small dimensions of the sensor unit 100 and short conductor lengths. In the case of very complex circuits, this technology often offers the only useful connection possibility, because otherwise several thousand contacts would have to be produced. In this way, the entire surface of the semiconductor component 130 can be used for contacting, by contrast to contacting methods that use bonding wires. However, such a contacting method is not possible and/or is possible only to a very limited extent, because the wires cross and are very likely to come into contact with one another. Furthermore, in contacting methods that use the bonding wires, the connections are produced one after the other. In the case of the flip chip assembly, all of the electrical contacts are connected simultaneously, an aspect that saves time.

FIG. 2 shows a section of a cross-section of a sensor unit 100 in accordance with one exemplary embodiment in a schematic detail view.

In the view of the sensor unit 100 shown here, the focus is on a diffusion barrier 210, in particular, by means of which the semiconductor element 130, the at least one contacting bump 160, and the substrate layer 140 are at least partially or completely coated in accordance with one exemplary embodiment. Such a coating ensures a medically qualified surface and provides protection for the electrical contacts from penetrating fluid and/or short circuits. In this embodiment, the medically qualified surface is shown only in the sensor unit 100, which also has a medium access 150.

In practice, the diffusion barrier is, for example, a layer of parylene C. Said layer is not in the form of a film, but rather is deposited directly onto the component, for example from the vapor phase in a vacuum. Therefore, in accordance with one exemplary embodiment, this diffusion barrier is not present as a component in a step of the method for producing the component, but rather, for example, is deposited from the vapor phase as the last production step in this exemplary embodiment. However, a separate laminating film or thin metallic membrane is, of course, also conceivable.

In accordance with one exemplary embodiment, the size of the recess also essentially matches the size of the semiconductor component 130 in the detail view of the sensor unit 100 shown here, as a result of which the semiconductor component 130 is integrated in the carrier material 110.

FIG. 3 shows a flowchart of one exemplary embodiment of a method 300 for producing a sensor unit in accordance with one exemplary embodiment. In accordance with one exemplary embodiment, the method 300 is executed and/or triggered on a device 310 for producing a sensor unit.

In a step 320, a carrier material, a semiconductor component, a substrate layer, and a diffusion barrier are provided. In a step 330 of the method 300, the carrier material, the semiconductor component, the substrate layer, and the diffusion barrier are arranged in such a way as to produce the sensor unit.

If an exemplary embodiment comprises an “and/or” conjunction between a first feature and a second feature, then such a conjunction should be understood to mean that the exemplary embodiment comprises both the first feature and the second feature in accordance with one exemplary embodiment and comprises either only the first feature or only the second feature in accordance with another embodiment.

1.-16. (canceled)

17. A sensor unit for a mechanical circulatory support device, the sensor unit comprising:
a carrier comprising a recess;
a semiconductor component configured to form a sensor, wherein the semiconductor component is positioned in the recess;
a substrate layer configured to cover at least a portion of the recess and comprising an opening; and
a diffusion barrier configured to cover at least a portion of the substrate layer and the semiconductor component, at least a portion of the diffusion barrier extending into a gap between the substrate layer and the semiconductor component.

18. The sensor unit of claim 17, wherein at least a portion of the substrate layer is coated with the diffusion barrier.

19. The sensor unit of claim 17, wherein the diffusion barrier comprises a fluid-tight coating or membrane preventing infusion of fluids.

20. The sensor unit of claim 17, further comprising a carrier material, wherein the carrier comprises at least one of: a metallic material, a thermoplastic material, a ceramic material, and glass.

21. The sensor unit of claim 17, further comprising at least one contacting element positioned between the semiconductor component and the substrate layer, the semiconductor component connected to the substrate layer via the at least one contacting element, the at least one contacting element comprising a gold material.

22. The sensor unit of claim 21, wherein a thickness of the at least one contacting element is within a range between 90 μm and 110 μm.

23. The sensor unit of claim 17, wherein the substrate layer is configured to be in direct contact with a tissue or a fluid of a patient, and wherein the substrate layer comprises at least one of: a polyimide element, a ceramic material, glass, and silicon.

24. The sensor unit of claim 17, wherein the recess is filled with silicone gel or silicone oil, and wherein the semiconductor component is partially surrounded by the silicone gel or the silicone oil.

25. The sensor unit of claim 17, wherein a size of the semiconductor component corresponds to a size of the recess.

26. The sensor unit of claim 17, wherein a thickness of the substrate layer is within a range between 40 μm and 60 μm.

27. The sensor unit of claim 17, wherein the sensor is a pressure sensor configured to detect an arterial pressure.

28. The sensor unit of claim 17, wherein the substrate layer is raised from the semiconductor component such that the opening of the substrate layer is positioned above the semiconductor component.

29. A method for manufacturing a sensor unit comprising:
placing a semiconductor component within a recess of a carrier;
covering at least a portion of the semiconductor component and at least a portion of the recess with a substrate layer, the substrate layer comprising an opening; and
placing a diffusion barrier over at least a portion of the substrate layer and at least a portion of the semiconductor component, at least a portion of the diffusion barrier extending into a gap between the substrate layer and the semiconductor component.

30. The method of claim 29, further comprising filling the recess with silicone gel or silicone oil, wherein the silicone gel or the silicone oil is configured to anchor the semiconductor component inside the recess.

31. The method of claim 29, wherein placing the diffusion barrier over at least the portion of the substrate layer and at least the portion of the semiconductor component comprises coating the semiconductor component and the substrate layer with the diffusion barrier.

32. The method of claim 29 further comprising:
positioning at least one contacting element between the semiconductor component and the substrate layer, wherein the semiconductor component is connected to the substrate layer via the at least one contacting element.

33. The method of claim 29, wherein the covering at least the portion of the semiconductor component and at least the portion of the recess with the substrate layer comprises positioning the substrate layer such that the substrate layer is raised form the semiconductor component and the opening of the substrate layer is positioned above the semiconductor component.

34. A system for manufacturing a sensor unit, the system comprising:
a processor;
a computer-readable storage medium storing therein computer-readable instructions that, when executed, cause the processor to:
place a semiconductor component within a recess of a carrier;
cover at least a portion of the semiconductor component and at least a portion of the recess with a substrate layer, the substrate layer comprising an opening; and
place a diffuser barrier over at least a portion of the substrate layer and at least a portion of the semiconductor component, at least a portion of the diffusion barrier extending into a gap between the substrate layer and the semiconductor component.

35. The system of claim 34, wherein the computer-readable instructions further cause the processor to:
fill the recess with silicone gel or silicone oil, wherein the silicone gel or the silicone oil is configured to partially surround the semiconductor component in the recess.

36. The system of claim 34, wherein the substrate layer is raised from the semiconductor component such that the opening of the substrate layer is positioned above the semiconductor component.
